(19) United States
(12) Patent Application Publication
(10) Pub. No.: US 2003/0134880 A1
(43) Pub. Date: Jul. 17, 2003

(54) Novel cd23 inhibitors

(76) Inventors: Gordon Bruton, Sawbridgeworth (GB); Barry Sidney Orlek, Essex (GB); Kishore Kalidas Rana, Welwyn Garden City (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/204,570

(22) PCT Filed: Feb. 23, 2001
(86) PCT No.: PCT/GB01/00807

(30) Foreign Application Priority Data
Feb. 24, 2000 (GB) 0004414.9
Dec. 21, 2000 (GB) 0031271.0

(51) Int. Cl. A61K 31/44; A61K 31/4172; A61K 31/381; A61K 31/34; A61K 31/165; C07D 333/66; C07D 233/80; C07D 213/56

(52) U.S. Cl. 514/357; 514/389; 514/443; 514/471; 514/424; 514/575; 546/336; 548/316.7; 549/57; 549/493; 562/621

(57) ABSTRACT

Compounds of formula (I) wherein R is hydrogen, alkyl, alkenyl, alkynyl, aryl, heteroaryl or heterocyclyl; R¹ is bicyclyl or heterobicyclyl; R² is aryl, heteroaryl, heterocyclyl, alkoxy, alkyl, hydroxy or optionally substituted amino and n is from 0 to 3; with the provisos than when n is 0 R² is alkyl or when n is from 1 to 3 R² is not alkyl, are useful in the treatment and prophylaxis of conditions mediated by CD23 or TNF.

This invention relates to novel inhibitors of the formation of soluble human CD23 and their use in the treatment of conditions associated with excess production of soluble CD23 (s-CD23) such as autoimmune disease, inflammation and allergy.

CD23 (the low affinity IgE receptor FceRII, Blast 2), is a 45 kDa type II integral protein expressed on the surface of a variety of mature cells, including B and T lymphocytes, macrophages, natural killer cells. Langerhans cells, monocytes and platelets (Delespesse et al, Adv Immunol, 49 [1991] 149-191). There is also a CD23-like molecule on eosinophils (Grangette et al, J Immunol, 143 [1989] 3580-3588). CD23 has been implicated in the regulation of the immune response (Delespesse et al, Immunol Rev, 125 [1992] 77-97). Human CD23 exists as two differentially regulated isoforms, a and b, which differ only in the amino acids at the intracellular N-terminus (Yokota et al, Cell, 55 [1988] 611-618). In man the constitutive a isoform is found only on B-lymphocytes, whereas type b, inducible by IL4, is found on all cells capable of expressing CD23.

Intact, cell bound CD23 (i-CD23) is known to undergo cleavage from the cell surface leading to the formation of a number of well-defined soluble fragments (s-CD23), which are produced as a result of a complex sequence of proteolytic events, the mechanism of which is still poorly understood (Bourget et al J Biol Chem. 269 [1994] 6927-6930). Although not yet proven, it is postulated that the major soluble fragments (Mr 37, 33, 29 and 25 kDa) of these proteolytic events, all of which retain the C-terminal lectin domain common to i-CD23, occur sequentially via initial formation of the 37 kDa fragment (Letellier et al, J Exp Med. 172 [1990] 693-700). An alternative intracellular cleavage pathway leads to a stable 16 kDa fragment differing in the C-terminal domain from i-CD23 (Grenier-Brosette et al, Eur J Immunol, 22 [1992] 1573-1577).

Several activities have been ascribed to membrane bound i-CD23 in humans, all of which have been shown to play a role in IgE regulation. Particular activities include: a) antigen presentation. b) IgE mediated eosinophil cytotoxicity, c) B cell homing to germinal centres of lymph nodes and spleen. and d) downregulation of IgE synthesis (Delespesse et al, Adv Immunol, 49, [1991] 149-191). The three higher molecular weight soluble CD23 fragments (Mr 37, 33 and 29 kDa) have multifunctional cytokine properties which appear to play a major role in IgE production. Thus, the excessive formation of s-CD23 has been implicated in the overproduction of IgE, the hallmark of allergic diseases such as extrinsic asthma, rhinitis, allergic conjunctivitis, eczema, atopic dermatitis and anaphylaxis (Sutton and Gould, Nature, 366, [1993] 421-428).

Other biological activities attributed to s-CD23 include the stimulation of B cell growth and the induction of the release of mediators from monocytes. Thus, elevated levels of s-CD23 have been observed in the serum of patients having B-chronic lymphocytic leukaemia (Sarfati et al. Blood, 71 [1988] 94-98) and in the synovial fluids of patients with rheumatoid arthritis (Chomarat et al, Arthritis and Rheumatism, 36 [1993] 234-242). That there is a role for CD23 in inflammation is suggested by a number of sources. First, sCD23 has been reported to bind to extracellular receptors which when activated are involved in cell-mediated events of inflammation. Thus, sCD23 is reported to directly activate monocyte TNF, IL-1, and IL-6 release (Armant et al, vol 180, J. Exp. Med., 1005-1011 (1994)). CD23 has been reported to interact with the B2-integrin adhesion molecules, CD11b and CD11c on monocyte/macrophage (S. Lecoanet-Henchoz et al, Immunity, vol 3; 119-125 (1995)) which trigger NO2- , hydrogen peroxide and cytokine (IL-1, IL-6, and TNF) release. Finally, IL4 or IFN induce the expression of CD23 and its release as sCD23 by human monocytes. Ligation of the membrane bound CD23 receptor with IgE/anti-IgE immune complexes or anti CD23 mAb activates cAMP and IL-6 production and thromboxane B2 formation, demonstrating a receptor-mediated role of CD23 in inflammation.

Because of these various properties of CD23, compounds which inhibit the formation of s-CD23 should have twofold actions of a) enhancing negative feedback inhibition of IgE synthesis by maintaining levels of i-CD23 on the surface of B cells, and b) inhibiting the immunostimulatory cytokine activities of higher molecular weight soluble fragments (Mr 37, 33 and 29 kDa) of s-CD23. In addition, inhibition of CD23 cleavage should mitigate sCD23-induced monocyte activation and mediator formation, thereby reducing the inflammatory response.

TNFα is a pro-inflammatory cytokine which is released from stimulated cells by specific cleavage of a 76-amino acid signal sequence in the inactive precursor to generate the mature form. The cleavage of TNFα has been reported to be carried out by a metalloprotease (Gearing, A. J. H. et al, (1994) Nature 370, 555-557; McGeehan, G. M. et al, (1994) Nature 370, 558-561; Mohler, K. M. et al, (1994) Nature 370, 218-220). Compounds reported to inhibit the cleavage of TNFα by the TNF processing enzyme can be broadly described as matrix metalloprotease inhibitors, particularly of the hydroxamic acid class.

TNFα is induced in a variety of cell types in response to bacteria, endotoxin, various viruses and parasites, so that one physiological function ascribed to TNFα is a contribution to the inflammatory response to acute infection by bacteria, parasites. etc (Dinarello, C. A. (1992) Immunol. 4, 133-145). Overproduction of TNFα has been implicated in disease states such as rheumatoid arthritis, septic shock, Crohn's disease and cachexia (Dinarello, 1992). Inhibition of processing of TNFα to the mature, active form would therefore be beneficial in the treatment of these inflammatory disorders. TNFα may also contribute to the destruction of tissue in autoimmune disease although it is not an initiating factor in these diseases. Confirming the importance of TNFα in rheumatoid arthritis, TNFα antibodies have been shown to reduce the severity of disease in short term studies in rheumatoid arthritis models (Elliott, M. J., et al (1993) Arthrit. Rheum. 12. 1681-1690; Elliott et al (1994) Lancet 344, 1125-1127).

International Patent Application No. WO 97/27174 (Shionogi & Co., Ltd) and International Patent Application number WO 95/35275 (British Biotech Ltd) disclose that certain compounds of formula (A):

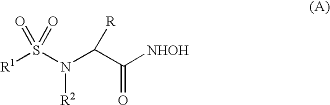

wherein

R¹ may be arylalkyl or heteroarylalkyl and R² is hydrogen or an organic substituent are effective inhibitors of mnetalloproteinases.

International Patent Application No. WO 98/46563 (British Biotech Ltd) discloses that certain compounds of formula (A) above in which R¹ may be phenylalkyl or heteroarylalkyl and R² is hydrogen or an organic substituent are effective inhibitors of matrix metalloproteases.

According to the present invention, there is provided a compound of formula (I):

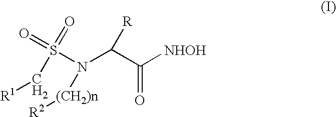

wherein

R is hydrogen, alkyl, alkenyl, alkynyl, aryl, heteroaryl or heterocyclyl; R¹ is bicyclyl or heterobicyclyl; R² is aryl, heteroaryl, heterocyclyl, alkoxy, alkyl, hydroxy, aryloxy or optionally substituted amino and n is from 0 to 3; with the provisos that when n is 0 R² is alkyl or when n is from 1 to 3 R² is not alkyl.

Amino referred to herein in the definition of the R² group includes amino groups substituted one or more times with (C1-6)alkyl.

Alkyl, alkenyl alkynyl and alkoxy groups referred to herein in the definition of the R and R² group include straight, branched and cyclic groups containing up to eight carbon atoms, and are optionally substituted by one or more groups selected from the group consisting of aryl, heterocyclyl, (C1-6)alkylthio, (C2-6)alkenylthio, (C2-6)alkynylthio, aryloxy, arylthio, heterocyclyloxy, heterocyclylthio, (C1-6)alkoxy, (C1-6)alkenyloxy, (C1-6)alkynyloxy, aryl(C1-6)alkoxy, aryl(C1 -6)alkylthio, amino, mono- or di-(C1-6)alkylamino, acylamino, sulfonylamino including C1-6 alkylsulfonylamino, aryl(C1-6)alkylsulfonylamino, aryl(C1-6)alkenylsulfonylamino and arylsulfonylamino, cycloalkyl, cycloalkenyl, carboxylic acid (C1-6) esters, hydroxy, halogen and carboxamide: CONR³R⁴ where R³ and R⁴ are independently selected from the group consisting of hydrogen, alkyl, aryl, arylalkyl and heterocyclyl, and includes R³ and R⁴ as part of a heterocyclyl group.

Cycloalkyl and cycloalkenyl groups referred to herein in the definition of the R and R² groups include groups having between three and eight ring carbon atoms and are optionally substituted as described hereinabove for alkyl, alkenyl and alkynyl groups.

When used herein in the definition of the R and R² groups, the term “aryl” includes phenyl. Suitably any aryl group, including phenyl, may be optionally substituted by up to five, preferably up to three substituents. Suitable substituents include halogen, CF₃, OCF₃, CN, (C₁₋₆)alkyl, (C₁₋₆)alkoxy, hydroxy, amino, heterocyclyl, heterocyclyl(C1-6)alkyl, mono- and di-N-(C1-6)alkylamino, acylamino, acyloxy, carboxy, (C1-6)alkoxycarbonyl, aminocarbonyl, mono- and di-N-(C1-6)alkylaminocarbonyl, mono- and di-N-(C1-6)alkylaminoalkyl, (C1-6)alkylsulfonylamino optionally substituted by alkyl, aminosulfonyl, mono- and di-N-(C1-6)alkylaminosulfonyl, (C1-6)mono- and dialkylaminosulfonyloxy, (C1-6)alkylsulfonyloxy, haloalkylsulfonyloxy including trifluoromethylsulfonyloxy, (C1-6)alkylthio and (C1-6)alkylsulfonyl. The term “aryl” includes single and fused rings, of which at least one is aromatic, which rings may be unsubstituted or substituted by, for example, up to three substituents as set out above. Each ring suitably has from 4 to 7, preferably 5 or 6, ring atoms.

When used herein in the definition of the R and R² groups the term “heteroaryl” suitably includes any heterocyclyl group which incorporates at least one aromatic ring (heterocyclic or carbocyclic). Suitable heteroaryl groups in the definition of the R group include thiophene, such as thiophen-2-yl and thiophen-3-yl. Suitable heteroaryl groups in the definition of the R² group include pyridyl such as 3-pyridyl and furyl such as furan-2-yl.

When used herein in the definition of the R and R² groups the terms “heterocyclyl” and “heterocyclic” suitably include, unless otherwise defined, aromatic and non-aromatic, single and fused, rings suitably containing up to four heteroatoms in each ring, each of which is selected from oxygen, nitrogen and sulphur, which rings, may be unsubstituted or substituted by, for example, up to three substituents. Each ring suitably has from 4 to 7, preferably 5 or 6, ring atoms. A fused heterocyclic ring system may include carbocyclic rings and need include only one heterocyclic ring. Preferably a substituent for a heterocyclyl group is selected from halogen, (C1-6)alkyl, (C1-6)alkoxy, hydroxy, CF₃, OCF₃, CN, amino, mono-and di-N-(C1-6)alkylamino, acylamino, acyloxy, carboxy, (C1-6)alkoxycarbonyl, aminocarbonyl, mono- and di-N-(C1-6)alkylaminocarbonyl, mono- and di-N-(C1-6)alkylaininoalkyl, (C1-6)alkylsulfonylamino. aminosulfonyl, (C1-6)alkylthio and (C1-6)alkylsulfonyl. Suitable heterocyclic groups include morpholino, succinimido, pyrrolidino, 2,5-dioxoimidazolidin-1-yl and N-pyrrolidone.

When used herein in the definition of the R¹ group “bicyclyl” means fused bicyclic rings suitably containing 4 to 7, preferably 5 or 6 ring atoms in each ring. One ring of the bicyclyl may be saturated or partially saturated. Suitable bicyclyl groups include naphthyl such as 2-naphthyl, tetrahydronaphthyl such as 2-tetrahydronaphthyl, and indanyl such as 2-indanyl.

When used herein in the definition of the R¹ group, heterobicyclyl means fused bicyclic aromatic and non-aromatic rings containing up to 4 heteroatoms in each ring, each of which is selected from oxygen, nitrogen and sulphur. Each ring suitably has from 4 to 7. preferably 5 or 6, ring atoms. The fused bicyclic ring system may include one carbocyclic ring and one of the rings may be saturated or partially saturated. Suitable heterobicyclyl groups include benzothiophene such as benzothiophen-5-yl and benzothiophen-6-yl.

Aromatic rings in bicyclyl and heterobicyclyl ring systems may be optionally substituted with up to three substituents. Suitable substituents include fluorine.

In a particular aspect of the invention, R is isobutyl, cyclohexyl or phenyl, and/or R¹ is 2-naphthyl, 5-benzothiophene, 2-tetrahydronaphthyl or 2-indanyl, and/or R² is 2-methoxyethoxy pyridin-3-yl, furan-2-yl, 5-dimethylaminofuran-2-yl, 2,5-dioxoimidazolidin-1-yl, 3-methyl-2,5-dioxoimidazolidin-1-yl, 3,4,4-trimethyl-2,5-dioxoimidazolidin-1-yl, N-morpholino, N,N-dimethylamino, N-succinimido, N-pyrrolidone or phenoxy and n is 1 or 2. In a further aspect of the invention, R, R1 ,R² and n are selected from the group consisting of the values ascribed to it in the Examples hereinbelow. Preferably, the compound of formula (I) of the invention is selected from the group consisting of the compounds described in the Examples hereinbelow.

According to a further aspect, the present invention provides the use of a compound of formula (I) for the production of a medicament for the treatment or prophylaxis of disorders such as allergy, inflammatory disorders, and autoimmune disease, in which the overproduction of s-CD23 is implicated.

In a further aspect the invention provides a method for the treatment or prophylaxis of disorders such as allergy, inflammatory disorders, and autoimmune disease, in which the overproduction of s-CD23 is implicated, which method comprises the administration of a compound of formula (I), to a human or non-human mammal in need thereof.

The invention also provides a pharmaceutical composition for the treatment or prophylaxis of disorders such as allergy, inflammatory disorders, and autoimmune disease, in which the overproduction of s-CD23 is implicated which comprises a compound of formula (I) and optionally a pharmaceutically acceptable carrier therefor.

Particular inflammatory disorders include CNS disorders such as Alzheimer's disease, multiple sclerosis, and multi-infarct dementia, as well as the inflammation mediated sequelae of stroke and head trauma.

According to a further aspect, the present invention provides the use of a compound of formula (I) for the production of a medicament for the treatment or prophylaxis of conditions mediated by TNF, including, but not limited to, inflammation, fever, cardiovascular effects, haemorrhage, coagulation and acute phase response, cachexia and anorexia, acute infections, shock states, graft versus host reactions and autoimmune disease.

In a further aspect the invention provides a method for the treatment or prophylaxis of conditions mediated by TNF, which method comprises the administration of a compound of formula (I), to a human or non-human mammal in need thereof.

The invention also provides a pharmaceutical composition for the treatment or prophylaxis of conditions mediated by TNF, which comprises a compound of formula (I) and optionally a pharmaceutically acceptable carrier therefor.

The present inventors have surprisingly found that the compounds of the invention are potent and selective inhibitors of both CD23 processing and TNF processing, whilst having little or no activity as inhibitors of matrix metalloproteases.

It is to be understood that the pharmaceutically acceptable salts, solvates and other pharmaceutically acceptable derivatives of the compound of formula (I) are also included in the present invention.

Salts of compounds of formula (I) include for example acid addition salts derived from inorganic or organic acids, such as hydrochlorides, hydrobromides, hydroiodides, p-toluenesulphonates, phosphates, sulphates, acetates, trifluoroacetates, propionates, citrates, maleates, fumarates, malonates, succinates, lactates, oxalates, tartrates and benzoates.

Salts may also be formed with bases. Such salts include salts derived from inorganic or organic bases, for example alkali metal salts such as sodium or potassium salts, and organic amine salts such as morpholine, piperidine, dimethylamine or diethylamine salts.

The compounds of the invention may be prepared by use of any appropriate conventional method, for example by analogy with the methods disclosed in patent publication EP-A-0 606 046.

Accordingly, a further aspect of the invention provides a process for preparing a compound of formula (I) as defined hereinabove, which process comprises

(a) deprotecting a compound of formula (II):

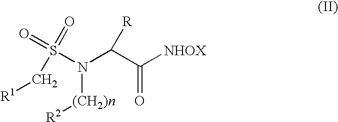

wherein

R, R¹, R² and n are as defined hereinabove, and X is a protecting group such as benzyl, t-butyldimethylsilyl or trimethylsilyl, or

(b) reacting a compound of formula (III):

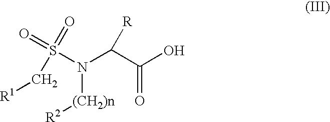

wherein

R, R¹, R² and n are as defined hereinabove, with hydroxylamine or a salt thereof, or

(c) converting a compound of formula (I) to a different compound of formula (I) as defined hereinabove.

Compounds of formula (II) and (III) are novel and form a further aspect of the invention.

Compounds of formula (III) can be prepared in accordance with Scheme 1 or Scheme 2:

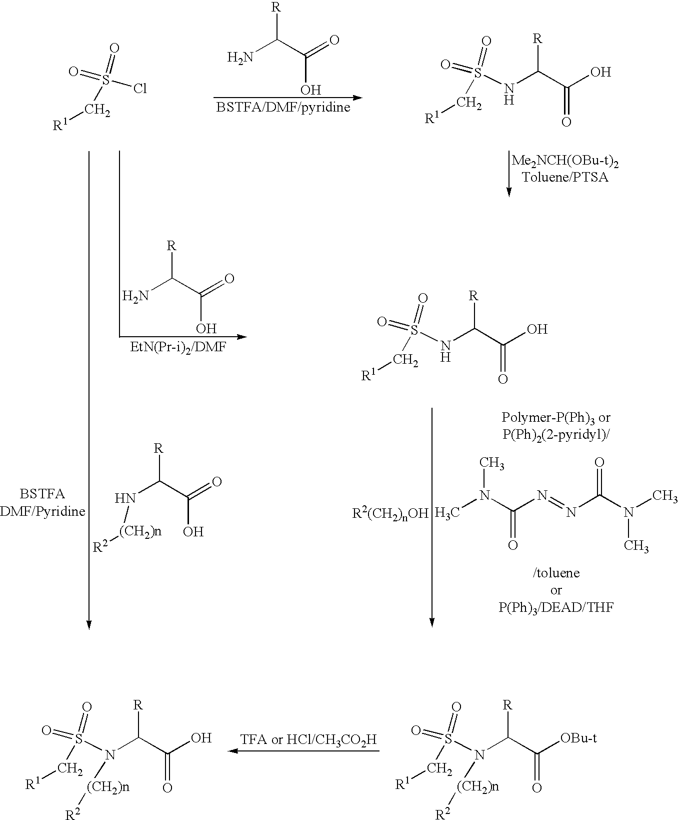

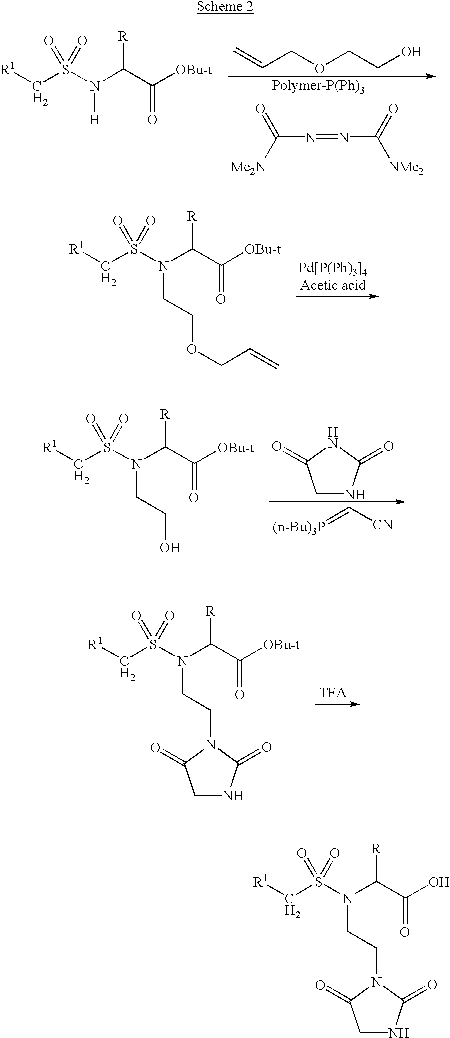

The process illustrated in Scheme 2 is also applicable to substituted imidazolidin-2,4-diones.

The starting materials and other reagents are available commercially or can be synthesised by well-known and conventional methods Sulfonylchlorides can be prepared by first reacting a compound of formula R¹-CH₂-Z wherein R¹ is as described hereinabove and Z is halogen or an alkyl or arylsulfonate with sodium sulfite to give the corresponding sodium sulfonate, which can optionally be converted by tetra-n-butyl ammonium hydrogen sulfate into the corresponding tetra-n-butylammonium sulfonate salt. The tetra-n-butylammonium sulfonate salt may be formed by direct conversion of a compound of formula R¹-CH-Z where Z is preferably bromide, chloride or iodide under phase transfer conditions. Conversion of the sulfonate salt into the sulfonyl chloride may be achieved using phosphorus oxychloride in acetonitrile and tetrahydrothiophene-1,1-dioxide at elevated temperature (Abdellaoui et al, Synth.Commun. 1995, 25(9) 1303). In the case of the tetra-n-butylammonium sulfonate the sulfonyl chloride is prepared using a chlorinating agent such as phosphorus pentachloride or triphosgene, preferably under low temperature conditions such as −20° C. or below, and preferably by addition of the sulfonate salt to the chlorinating agent.

The isomers, including stereoisomers, of the compounds of the present invention may be prepared as mixtures of such isomers or as individual isomers. The individual isomers may be prepared by any appropriate method, for example individual stereoisomers may be prepared by stereospecific chemical synthesis starting from chiral substrates or by separating mixtures of enantiomers or mixtures of diastereoisomers using known methods. In a preferred aspect, the invention provides compounds of formula (IA):

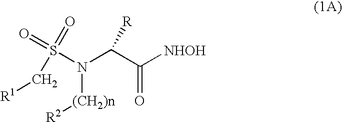

It is preferred that the compounds are isolated in substantially pure form.

As stated herein an inhibitor of the formation of soluble human CD23 has useful medical properties. Preferably the active compounds are administered as pharmaceutically acceptable compositions.

The compositions are preferably adapted for oral administration. However, they may be adapted for other modes of administration, for example in the form of a spray, aerosol or other conventional method for inhalation, for treating respiratory tract disorders; or parenteral administration for patients suffering from heart failure. Other alternative modes of administration include sublingual or transdermal administration.

The compositions may be in the form of tablets, capsules, powders, granules, lozenges, suppositories, reconstitutable powders, or liquid preparations, such as oral or sterile parenteral solutions or suspensions.

In order to obtain consistency of administration it is preferred that a composition of the invention is in the form of a unit dose.

Unit dose presentation forms for oral administration may be tablets and capsules and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulphate.

The solid oral compositions may be prepared by conventional methods of blending, filling or tabletting. Repeated blending operations may be used to distribute the active agent throughout those compositions employing large quantities of fillers. Such operations are of course conventional in the art. The tablets may be coated according to methods well known in normal pharmaceutical practice, in particular with an enteric coating.

Oral liquid preparations may be in the form of, for example, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

For parenteral administration, fluid unit dosage forms are prepared utilising the compound and a sterile vehicle, and, depending on the concentration used, can be either suspended or dissolved in the vehicle. In preparing solutions the compound can be dissolved in water for injection and filter sterilised before filling into a suitable vial or ampoule and sealing. Advantageously, adjuvants such as a local anaesthetic, a preservative and buffering agents can be dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspending in the sterile vehicle. Advantageously, a surfactant or wetting agent is included in the composition to facilitate uniform distribution of the compound.

Compositions of this invention may also suitably be presented for administration to the respiratory tract as a snuff or an aerosol or solution for a nebulizer, or as a microfine powder for insufflation, alone or in combination with an inert carrier such as lactose. In such a case the particles of active compound suitably have diameters of less than 50 microns, preferably less than 10 microns for example diameters in the range of 1-50 microns, 1-10 microns or 1-5 microns. Where appropriate, small amounts of other anti-asthmatics and bronchodilators, for example sympathomimetic amines such as isoprenaline, isoetharine, salbutamol, phenylephrine and ephedrine; xanthine derivatives such as theophylline and aminophylline and corticosteroids such as prednisolone and adrenal stimulants such as ACTH may be included.

The compositions may contain from 0.1% to 99% by weight, preferably from 10-60% by weight, of the active material, depending upon the method of administration. A preferred range for inhaled administration is 10-99%, especially 60-99%, for example 90, 95 or 99%.

Microfine powder formulations may suitably be administered in an aerosol as a metered dose or by means of a suitable breath-activated device.

Suitable metered dose aerosol formulations comprise conventional propellants, cosolvents, such as ethanol, surfactants such as oleyl alcohol, lubricants such as oleyl alcohol, desiccants such as calcium sulphate and density modifiers such as sodium chloride.

Suitable solutions for a nebulizer are isotonic sterilised solutions, optionally buffered, at for example between pH 4-7, containing up to 20 mg/ml of compound but more generally 0.1 to 10 mg/ml. for use with standard nebulisation equipment.

An effective amount will depend on the relative efficacy of the compounds of the present invention, the severity of the disorder being treated and the weight of the sufferer. Suitably, a unit dose form of a composition of the invention may contain from 0.1 to 1000 mg of a compound of the invention (0.001 to 10 mg via inhalation) and more usually from 1 to 500 mg, for example 1 to 25 or 5 to 500 mg. Such compositions may be administered from 1 to 6 times a day, more usually from 2 to 4 times a day, in a manner such that the daily dose is from 1 mg to 1 g for a 70 kg human adult and more particularly from 5 to 500 mg. That is in the range of about 1.4×10⁻² mg/kg/day to 14 mg/kg/day and more particularly in the range of about 7×10⁻² mg/kg/day to 7 mg/kg/day.

The following examples illustrate the invention but do not limit it in any way.

### BIOLOGICAL TEST METHODS

Procedure 1: The ability of test compounds to inhibit the release of soluble CD23 was investigated by use of the following procedure.

RPMI 8866 Cell Membrane CD23 Cleavage Activity Assay:

Plasma membranes from RPMI 8866 cells, a human Epstein-Barr virus transformed B-cell line (Sarfati et al., Immunology 60 [1987] 539-547) expressing high levels of CD23 are purified using an aqueous extraction method. Cells resuspended in homogenisation buffer (20 mM HEPES pH 7.4, 150 mM NaCl, 1.5 mM MgCl2. 1 mM DTT) are broken by N2 cavitation in a Parr bomb and the plasma membrane fraction mixed with other membranes is recovered by centrifugation at 10,000×g. The light pellet is resuspended in 0.2 M potassium phosphate, pH 7.2 using 2 ml per 1-3 g wet cells and the nuclear pellet is discarded. The membranes are further fractionated by partitioning between Dextran 500 (6.4% w/w) and polyethylene glycol (PEG) 5000 (6.4% w/w), at 0.25 M sucrose in a total of 16 g per 10-15 mg membrane proteins [Morre and Morre, BioTechniques 7, 946-957 (1989)]. The phases are separated by brief centrifugation at 1000×g and the PEG (upper) phase is collected, diluted 3-5 fold with 20 mM potassium phosphate buffer pH 7.4, and centrifuged at 10,000×g to recover membranes in that phase. The pellet is resuspended in phosphate-buffered saline and consists of 3-4 fold enriched plasma membranes as well as some other cell membranes (e.g. lysosomes, Golgi). The membranes are aliquoted and stored at −80° C. Fractionation at 6.6% Dextran/PEG yields plasma membranes enriched 10-fold.

The fractionated membranes are incubated at 37° C. for times up to 4 hrs to produce fragments of CD23 which are separated from the membrane by filtration in 0.2 micron Durapore filter plates (Millipore) after quenching the assay with 5 uM Preparation 1 from WO 95/11457 ([4-(n-Hydroxyamino)-2-(R)-isobutyl-3-(S)-(2-thiophenethiomethyl)succinyl]-(S)-phenylalanine-N-methylamide sodium salt, prepared according to the procedure described in Example 11 of WO 90/05719). sCD23 released from the membrane is determined using the EIA kit from The Binding Site (Birmingham, UK) or a similar one utilising MHM6 anti-CD23 mAb [Rowe et al., Int. J. Cancer, 29, 373-382 (1982)] or another anti-CD23 mAb as the capture antibody in a sandwich EIA.. The amount of soluble CD23 made by 0.5 ug membrane protein in a total volume of 50 ul phosphate-buffered saline is measured by EIA and compared to the amount made in the presence of various concentrations of inhibitors. Inhibitors are prepared in solutions of water or dimethylsulfoxide (DMSO) and the final DMSO concentration is not more than 2%. IC50's are determined by curve fitting as the concentration where 50% inhibition of production of sCD23 is observed relative to the difference in sCD23 between controls incubated without inhibitor.

Results

The compounds of the Examples all showed IC₅₀ values≦1 μM.

Procedure 2: The ability of test compounds to inhibit collagenase was investigated using the following procedure.

Collagenase Inhibition Assay:

The potency of compounds to act as inhibitors of collagenase was determined by the method of Cawston and Barrett (Anal. Biochem. 99, 340-345, 1979), hereby incorporated by reference, whereby a 1 mM solution of the inhibitor being tested or dilutions thereof, was incubated at 37° C. for 18 h with collagen and human recombinant collagenase, from synovial fibroblasts cloned, expressed and purified from E. Coli, (buffered with 150 mM Tris, pH 7.6. containing 15 mM calcium chloride, 0.05% Brij 35.200 mM sodium chloride and 0.02% sodium azide). The collagen was acetylated ³H type 1 bovine collagen prepared by the method of Cawston and Murphy (methods in Enzymology 80. 711,1981) The samples were centrifuged to sediment undigested collagen and an aliquot of the radioactive supernatant removed for assay on a scintillation counter as a measure of hydrolysis. The collagenase activity in the presence of 1 mM inhibitor, or dilution thereof, was compared to activity in a control devoid of inhibitor and the results reported as that concentration effecting 50% of the collagenase (IC₅₀).

Results

The compounds of Examples 1, 2, 8 and 14 showed IC₅₀ values≦10 μM.

Preparation of Intermediates

Preparation 1: Naphthalen-2-ylmethanesulfonyl chloride

Step 1: Sodium naphthalen-2-ylmethanesulfonate—2-Bromomethyl-naphthalene (70 g),was dissolved in dioxan(350 ml) and treated with sodium sulfite (240 g) in water (500 ml). The mixture was heated under reflux for 30 min. On cooling a white solid was obtained which was filtered off, washed with ether and dried to give the subtitle methanesulfonate salt (69 g).

Step 2: Naphthalen-2-ylmethanesulfonyl chloride—To sodium naphthalen-2-ylmethanesulfonate (12 g) in tetrahydrothiophene-1,1-dioxide (96 ml) were added acetonitrile (48 ml) and phosphorus oxychloride (24 ml) and the mixture was heated. When the internal temperature reached 100° C. unreacted starting material was filtered off and the hot filtrate was poured onto ice. A brown solid was filtered off and washed with hexane to give title compound (5.5 g).

Preparation 2: Benzo[b]thiophene-5-methanesulfonyl chloride

Step 1: 5-Bromomethylbenzo[b]thiophene—A solution containing 5-methylbenzo[b]thiophene (37 g), N-bromosuccinimide (46 g), and tetrachloromethane (400 ml) was refluxed for 4 h, cooled, and filtered. The filtrate was evaporated and the resultant residue crystallised from hexane to give the subtitle compound (40 g).

Step 2: Tetra-n-butylammonium benzo[b]thiophene-5-methanesulfonate—A mixture containing 5-bromomethylbenzo[b]thiophene (40 g), tetra-n-butylammonium hydrogen sulfate (135 g), sodium hydroxide (14 g), sodium sulfite (45 g), dichloromethane (300 ml), and water (300 ml) was stirred vigorously overnight. The organic layer was dried (MgSO₄), evaporated, dissolved in THF (130 ml), re-evaporated, and dissolved again in THF (130 ml). Addition of ether (200 ml) gave the crystalline subtitle compound containing an equimolar amount of tetra-n-butylammonium bromide (132 g).

Step 3: Benzo[b]thiophene-5-methanesulfonyl chloride—A solution of the tetra-n-butylammonium benzo[b]thiophene-5-methanesulfonate from step 2 (30 g) in dichloromethane (150 ml) was added to a cooled suspension of phosphorus pentachloride (8.3 g) in dichloromethane (150 ml) at an internal temperature of −20° C. The solution was warmed to room temperature and maintained at room temperature for 15 min, then filtered through a pad of silicagel washing with ethyl acetate:hexane (1:1). The combined eluates were dissolved in toluene and the resulting solution again filtered through silica gel, eluting with more toluene. Evaporation of the eluate and crystallization from hexane gave the title compound (7.5 g). ¹H NMR δ(CDCl₃) 7.95(1H,d,J 8 Hz), 7.94(1H,s), 7.54(1H,d,J 6 Hz), 7.43(1H,d,J 8 Hz), 7.38(1H,d,J 6 Hz), 4.99(2H,s). In like manner was prepared naphthalene-2-methanesulfonyl chloride from 2-bromomethylnaphthalene.

### EXAMPLE 1

### (R)-2-{(Benzo[b]thiophen-5-ylmethanesulfonyl)[2-(2-methoxyethoxy)-ethyl]amino}-N-hydroxy-2-phenylacetamide

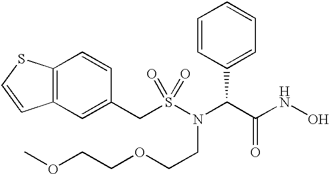

Step 1: (R)-2-(Benzo[b]thiophen-5-ylmethanesulfonylamino)-2-phenlacetic acid—A suspension of (R)-phenylglycine (0.38 g) in pyridine (5 ml) and DMF (5ml) at 55° C. was treated with BSTFA (1.35 ml). After 30 min at 55° C. the solution was cooled to 0° C. and a solution of benzo[b]thiophene-5-methanesulfonyl chloride (0.75 g) in DMF (2 ml) was added. After a further 2h at rt ethyl acetate and aqueous potassium hydrogen sulphate were added, and the organic layer was dried (MgSO₄) and evaporated. Crystallisation of the residue from ether gave the subtitle compound (0.40 g).

Step 2: (R)-2-(Benzo[b]thiophen-5-ylmethanesulfonylamino)-2-phenylacetic acid-tert-butyl ester—(R)-2-(Benzo[b]thiophen-5-ylmethanesulfonylamino)-2-phenylacetic acid (1.13 g) was heated at reflux for 6 h in toluene (30 ml) containing N,N-dimethylformamide di-tert-butyl acetal (5 ml) with a catalytic quantity of p-toluenesulfonic acid. The reaction was then washed with a saturated sodium hydrogen carbonate solution and brine. Evaporation of toluene and trituration with n-hexane and ether gave the subtitle compound (0.95 g).

Step 3: (R)-2-{(Benzo[b]thiophen-5-ylmethanesulfonyl)[2-(2-methoxyethoxy)ethyl]-amino}-2-phenylacetic acid tert-butyl ester—(R)-2-(Benzo[b]thiophen-5-ylmethanesulfonylamino)-2-phenylacetic acid-tert-butyl-ester (0.15 g) in toluene (3 ml) containing diphenyl-2-pyridylphosphine (0.1 89 g), N,N,N′,N′-tetramethylazodicarboxamide (0.124 g) and 2-(2-methoxyethoxy)ethanol (0.066 ml) was heated to 70° C. for 3 h. The reaction was cooled, washed with citric acid then brine and evaporated to a gum. Trituration with n-hexane afforded the subtitle compound contaminated with 2-(2-methoxyethoxy)ethanol.

Step 4: (R)-2-{(Benzo[b]thiophen-5-ylmethanesulfonyl)-12-(2-methoxyethoxy)-ethyl]amino}-2-phenylacetic acid—The crude product from Step 3 was dissolved in trifluoroacetic acid (5 ml) and after 2 h evaporated from toluene (3×20 ml) and toluene/water (2:1) (3×30 ml). The residue was triturated with n-hexane and with ether to give the subtitle compound (0.12 g).

Step 5: (R)-2-{(Benzo[b]thiophen-5-ylmethanesulfonyl)-[2-(2-methoxyethoxy)-ethyl]amino}-N-hydroxy-2-phenylacetamide—(R)-2-{(Benzo[b]thiophen-5-ylmethanesulfonyl)-[2-(2-methoxyethoxy)ethyl]amino}-2-phenylacetic acid (0.13 g) in DMF (2 ml) was treated with HOAT (0.039 g) and EDC (0.108 g). After 10 min this solution was added over 0.75 h to a mixture of hydroxylamine hydrochloride (0.097 g) and N-methylmorpholine (0.155 ml) in DMF (2 ml) which had been stirring for 15 min. After 20 h the reaction was diluted with ethyl acetate and water (20 ml) and the organic layer isolated and passed through an SCX column. The solution was then passed through an SAX column which was washed with further ethyl acetate before eluting with methanol to give the title compound (0.082 g). MS electrospray (+ve ion) 501 (MNa⁺) and 479 (MH⁺), (−ve ion) 477 (M−H⁻), ¹H NMR δ (CD₃OD) 7.25-7.89 (10H,m), 5.76 (1H,s), 4.95 and 4.43 (2H, ABq, J=8.8 Hz), 3.69 (1H, m), 3.49-3.55 (3H, m), 3.34-3.39 (3H, m), 3.24-3.28 (1H,m) and 2.9-2.99 (1H, m).

### EXAMPLE 2

### (R,S)-2-[(Benzo[b]thiophen-5-ylmethanesulfonyl)(pyridin-3-ylmethyl)-amino]-N-hydroxy-2-phenylacetamide

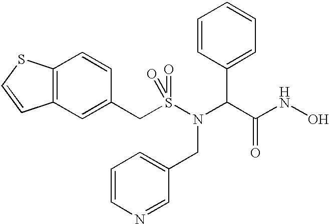

Step 1: (R,S)-2-[(Benzo[b]thiophen-5-ylmethanesulfonyl)(pyridin-3-ylmethyl)-amino]-2-phenylacetic acid-tert-butyl-ester—(R,S)-2-(Benzo[b]thiophen-5-ylmethanesulfonylamino)-2-phenylacetic acid tert-butyl ester (0.15 g), prepared as for Example 1, Step 2, was heated for 4 h at 85° C. in toluene (4 ml) containing 3-pyridylmethanol (0.22 ml), resin bound triphenylphosphine (0.5 g, 3 mmol/g) and N,N,N′,N′-tetramethylazodicarboxamide (0.125 g). The reaction was then filtered through celite and passed through a Bond Elut PSA column eluting with ethyl acetate/hexane (1:1). Evaporation of the eluent gave the subtitle compound used directly in the following step.

Step 2: (R,S)-[(Benzo[b]thiophen-5-ylmethanesulfonyl)(pyridin-3-ylmethyl)amino]-2-phenylacetic acid—The crude product from Step 1 was dissolved in 95% trifluoroacetic acid/water (5 ml) and after 4 h evaporated from toluene (3×20 ml) and toluene/water (2:1) (3×30 ml). The product was captured on a Bond Elut PSA column. washed with ethyl acetate, methanol and once more with ethyl acetate. The product was eluted from the column with 4% TFA/water. Evaporation and then re-evaporation from toluene gave the subtitle compound (0.12 g).

Step 3: (R,S)-2-[(Benzo[b]thiophen-5-ylmethanesulfonyl)(pyridin-3-ylmethyl)-amino]-N-hydroxy-2-phenylacetamide—(R,S)-[(Benzo[b]thiophen-5-ylmethanesulfonyl)(pyridin-3 -ylmethyl)amino]-2-phenylacetic acid (0.12 g) was treated with HOAT (0.037 g) and EDC (0.095 g). After 10 min this solution was added over 0.75 h to a mixture of hydroxylamine hydrochloride (0.097 g) and N-methylmorpholine (0.155 ml) in DMF (2 ml) which had been stirring for 15 min. After 20 h the reaction was diluted with ethyl acetate and water (20 ml) and the organic layer isolated and passed through an SAX column which was washed with further ethyl acetate before eluting with methanol. Evaporation of the organics and chromatography (silica gel, step gradient methanol/DCM) gave the title compound (0.04 g). MS electrospray (+ve ion) 468 (MH⁺),¹H NMR δ (CD₃OD) 7.17-8.27 (14H, m), 5.65 (1H, s), 4.78 and 4.72 (2H, ABq, J=10.4 Hz) and 4.63 and 4.57 (2H, ABq, J=8.6 Hz).

The following examples were prepared by the procedures described in Example 1 and Example 2. ¹H NMR and mass spectra were consistent with the structures given in the table.

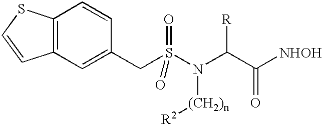

| Example No | R | R²(CH₂)ₙ |
|---|---|---|
| 3 | 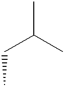 | 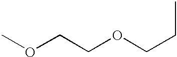 |
| 4 | 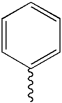 | 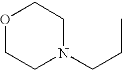 |
| 5 | 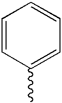 | 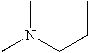 |
| 6 | 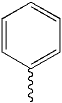 | 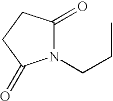 |
| 7 | 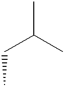 | 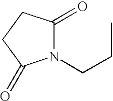 |
| 8 | 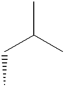 | 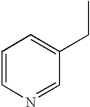 |
| 9 | 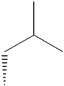 | 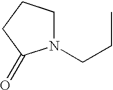 |
| 10 | 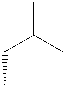 | 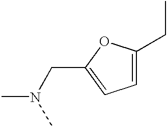 |
| 11 | 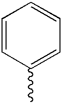 | 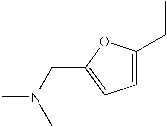 |
| 12 | 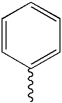 | 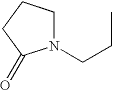 |
| 13 | 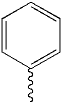 | 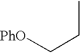 |

### EXAMPLE 14

### (R)-2-[(Benzo[b]thiophen-5-ylmethanesulfonyl)(furan-2-ylmethyl)-amino]-2-cyclohexyl-N-hydroxy-acetamide

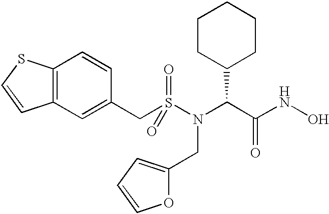

Step 1: (R)-2-Cyclohexyl-2-[(furan-2-ylmethyl)amino]acetic acid—(R)-cyclohexylglycine (0.95 g) in DMF (15 ml) was treated with BSTFA (2 ml) and 2-furyl aldehyde (0.5 ml). After 3 h NaBH₃CN on resin (0.7 g, 3.64 mmol/g) was added and the reaction stirred for 18 h. The reaction was filtered and concentrated, and re-evaporated from toluene (3×). The residue was treated with methanol (20 ml) and the precipitate filtered off to give the subtitle compound (0.4 g).

Step 2: (R)-[(Benzo[b]thiophen-5-ylmethanesulfonyl)(furan-2-ylmethyl)amino]-2-cyclohexylacetic acid—(R)-2-Cyclohexyl-2-[(furan-2-ylmethyl)amino]acetic acid (0.28 g) was treated as for Example 1, Step 1 to give the subtitle compound (0.18 g).

Step 3: (R)-2-[(Benzo[b]thiophen-5-ylmethanesulfonyl)(furan-2-ylmethyl)amino]-2-cyclohexyl-N-hydroxy-acetamide—(R)-[(Benzo[b]thiophen-5-ylmethanesulfonyl)(furan-2-ylmethyl)amino]-2-cyclohexylacetic acid (0.18 g) in DCM (2.5 ml) at 0° C. under argon was treated with oxalyl chloride (0.038 ml) and DMF (0.035 ml). After 30 min this reaction was added to a THF solution of hydroxylamine, prepared from hydroxylamine-HCl (0.111 g) and diethylaminoethyl polystyrene resin (1.6 g, 3.2 mmol/g) in THF (5 ml) and water (1 ml) at 0° C. After 18 h the reaction was diluted with ethyl acetate and washed with dilute HCl and brine. The product, after evaporation, was chromatographed (silica gel, step gradient methanol/DCM) to give the title compound (0.024 g). MS electrospray (+ve ion) 463 (MH⁺), 925 (2MH⁺); MS electrospray (−ve ion) 461 (M−H⁻), 923 (2M−H⁻). ¹H NMR (CD₃OD) δ7.23-7.8 (6H, m), 6.27-6.3 (2H, m), 4.73 and 4.26 (2H, ABq, J=10.2 Hz), 4.21 and 4.13 (2H ABq, J=8.5 Hz), 3.7 (1H, d, J=6.8 Hz) and 0.61-1.89(11H, m).

### EXAMPLE 15

### (R)-N-Hydroxy-4-methyl-2-1(naphthalen-2-ylmethanesulfonyl)(pyridin-3-ylmethyl)amino]pentanoic acid amide

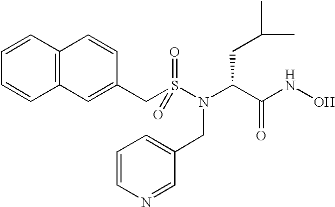

Step 1: (R)-4-Methyl-2-(naphthalen-2-ylmethanesulfonylamino)pentanoic acid tert-butyl ester—(D)-Leucine tert-buyl ester hydrochloride (0.47 g) in dry DMF (3 ml) was treated with diisopropylethylamine (0.72 ml) followed by a solution of naphthalen-2-ylmethanesulfonyl chloride (0.5 g) in dry DMF (1.5 ml). After stirring at rt for 2 h the reaction mixture was concentrated to dryness and the residue was partitioned between ethyl acetate and water. The organic layer was dried (MgSO₄) and concentrated and the residue was purified by flash chromatography (silica gel, step gradient 2-40% ethyl acetate/hexane) to give the subtitle compound as a white solid (0.7 g).

Step 2: (R)-4-Methyl-2-[(naphthalen-2-ylmethanesulfonyl)(pyridin-3-ylmethyl)amino]pentanoic tert-butyl ester—A solution of (R)-4-methyl-2-(naphthalen-2-ylmethanesulfonylamino)pentanoic acid tert-butyl ester (0.2 g), triphenylphosphine (0.4 g) and 3-pyridylmethanol (0.15 ml) in dry THF (6 ml) was cooled to 0° C. and treated dropwise with diethyl azodicarboxylate (0.25 ml). The mixture was stirred at 0° C. for a further 5 min and then warmed to room temperature and left stirring overnight. Further aliquots of triphenylphosphine (0.268 g) and 3-pyridylmethanol (0.1 ml) were added and the mixture was then treated dropwise at 0° C. with diethyl azodicarboxylate (0.167 ml). The reaction was allowed to warm to rt and then left stirring for 2 h. The mixture was absorbed on silica gel and purified by chromatography (silica gel, step gradient 2-60% ethyl acetate/hexane) to give the subtitle compound as a white solid (0.17 g).

Step 3: (R)-4-Methyl-2-[(naphthalen-2-ylmethanesulfonyl)(pyridin-3-ylmethyl)amino]pentanoic acid—(R)-4-Methyl-2-[(naphthalen-2-ylmethanesulfonyl)(pyridin-3-ylmethyl)amino]pentanoic tert-butyl ester (0.17 g) was taken up in acetic acid (6 ml), treated with 4M HCl (3 ml) and left stirring overnight. The mixture was concentrated to dryness (bath temperature 50° C.) and the residue was was triturated with ether to give the subtitle compound as a white solid (0.095 g).

Step 4: (R)-N-(tert-Butyldimethylsilyloxy-4-methyl-2-[(naphthalen-2-ylmethanesulfonyl)(pyridin-3-ylmethyl)amino]pentanoic acid amide—A solution of (R)-4-methyl-2-[(naphthalen-2-ylmethanesulfonyl)(pyridin-3-ylmethyl)amino]pentanoic acid (25 mg) in dry DCM (1 ml) was cooled to 0° C. and treated with O-(tert-butyldimethylsilyl)hydroxylamine (13 mg) in dry DCM (0.5 ml) followed by EDC methiodide (26 mg) in dry DCM (0.5 ml). The reaction mixture was allowed to warm slowly to rt and after standing overnight the reaction was concentrated to dryness and the residue was partitioned between ethyl acetate and saturated aqueous sodium bicarbonate. The organic layer was washed with brine, dried (Na₂SO₄) and concentrated. The residue was dissolved in hexane/ethyl acetate (1:1) and purified by filtration through a plug of silica gel eluting with 1:1 hexane/ethyl acetate (75 ml) to give the subtitle compound (15 mg).

Step 5: (R)-N-Hydroxy-4-methyl-2-[(naphthalen-2-ylmethanesulfonyl)(pyridin-3-ylmethyl)amino]pentanoic acid amide—A solution of N-(tert-butyldimethylsilyloxy-4-methyl-2-[(naphthalen-2-ylmethanesulfonyl)(pyridin-3 -ylmethyl)amino]pentanoic acid amide (15 mg) in dry THF (0.5 ml) was treated with TBAF (0.04 ml of a 1M solution in THF) and left stirring for 30 min. The reaction was concentrated to dryness, and the residue purified by preparative HPLC (reverse phase C18 column, gradient 0.1% TFA/H₂O -0.1% TFA/CH₃CN) to give the title compound (4 mg). MS electrospray (+ve ion) 442 (M+H⁺). ¹H NMR δ (CD₃OD): 8.50 (1H, s), 8.2 (2H, m), 7.79−7.66 (4H, m), 7.48−7.34 (4H, m), 4.53 (2H, s), 4.51 (2H, s), 4.18−4.12 (1H, m), 1.74−1.65 (1H, m), 1.28−1.13 (2H, m), 0.75 (3H, d, J=6.2 Hz), 0.66 (3H, d, J=6.2 Hz).

### EXAMPLE 16

### (R)-2-{(Benzo[b]thiophen-5-ylmethanesulfonyl)[2-(2,5-dioxo-imidazolidin-1-yl)ethyl]amino}-4-methyl-N-hydroxy-pentanamide

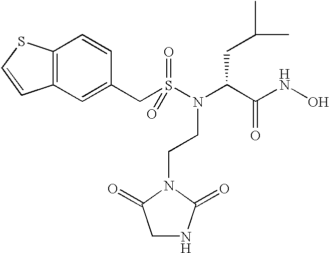

Step 1: (R)-2-(Benzo[b]thiophene-5-ylmethanesulfonylamino)-4-methyl-pentanoic acid tert-butyl ester—(D)-Leucine tert-buyl ester hydrochloride (0.906 g) was stirred with BSTFA (3 ml) in dry DMF/pyridine (1:1 by vol; 6 ml) at rt until homogeneous. The reaction was then cooled to 0° C. and benzo[b]thiophen-5-ylmethanesulfonyl chloride (1.00 g) was added. The solution was stirred at 0° C. for 1 h and then stirred at rt overnight. The reaction mixture was dissolved in ethyl acetate and washed with 1 N HCl and water. The organic layer was evaporated to a brown crude which was chromatographed (silica-gel, step gradient 0-10% ethyl acetate/hexane) to yield the subtitle compound as a white solid (0.897 g)

Step 2: (R)-2-1(2-Allyloxyethyl)(benzo[b]thiophen-5-ylmethanesulfonyl)-amino]-4-methyl-pentanoic acid tert butyl ester—(R)-2-(Benzo[b]thiophene-5-ylmethanesulfonylamino)-4-methyl-pentanoic acid tert-butyl ester (0.695 g) was stirred in dry toluene (15 ml) with 2-allyloxyethanol (0.41 ml) and triphenyl phosphine resin (3 mmol/g loading, 1.739 g) under an argon atmosphere. N,N,N′,N′-tetramethylazodicarboxamide (0.662 g) was then added portionwise and the reaction heated to 65° C. for 3 h and then stirred at rt overnight. The reaction mixture was filtered through celite and the filtrate evaporated to a brown crude which was chromatographed (silica-gel, step gradient 0-10% ethyl acetate/hexane) to yield the subtitle product as a clear oil (0.702 g).

Step 3: (R)-2-[(Benzo[b]thiophen-5-ylmethanesulfonyl)(2-hydroxyethyl)-amino]-4-methylpentanoic acid tert-butyl ester—(R)-2-[(2-Allyloxyethyl)-(benzo[b]thiophen-5-ylmethanesulfonyl)amino]-4-methylpentanoic acid tert butyl ester (0.702 g) and tetrakis(triphenylphosphine)palladium (0.36 g) were stirred in acetic acid (15 ml) under argon at 80° C. for 3 h and then left to stand at rt overnight. The reaction mixture was concentrated, and the crude product was chromatographed (silica-gel, step gradient 0-20% ethyl acetate/hexane) to yield the subtitle compound as a yellow oil (0.398 g).

Step 4: (R)-2-{(Benzo [b]thiophen-5-ylmethanesulfonyl)[2-(2,5-dioxo-imidazolidin 1-yl)ethyl]amino}-4-methylpentanoic acid tert-butyl ester—(R)-2-[(Benzo[b]thiophen-5-ylmethanesulfonyl)(2-hydroxyethyl)amino]-4-methyl-pentanoic acid tert-butyl ester (0.217 g) was stirred in dry benzene (3 ml) with imidazolidin-2.4-dione (0.073 g) under argon. Cyanomethylenetri-n-butylphosphorane (0.178 g) was dissolved in dry benzene (1 ml) and added, dropwise, to the stirring mixture which was heated to 100° C. for 3 h. The reaction mixture was concentrated and then chromatographed (silica-gel, step gradient 1-50% ethyl acetate/hexane) to yield the subtitle compound as a white solid (0.074g)

Step 5: (R)-2-{(Benzo[b]thiophen-5-ylmethanesulfonyl)[2-(2,5-dioxo-imidazolidin-1-yl)ethyl]amino}-4-methylpentanoic acid—(R)-2-{(Benzo[b]thiophen-5-ylmethanesulfonyl)[2-(2,5-dioxo-imidazolidin-1-yl)ethyl]-amino}-4-methylpentanoic acid tert-butyl ester (0.074 g) was stirred in TFA/water (95% TFA, 10 ml) for 2 h. The reaction mixture was then evaporated and co-evaporated with toluene to yield the subtitle compound as a yellow oil (0.062 g).

Step 6: (R)-2-{(Benzo[b]thiophen-5-ylmethanesulfonyl)[2-(2,5-dioxo-imidazolidin-1-yl)ethyl]amino}-4-methyl-N-hydroxy-pentanamide—(R)-2-{(Benzo[b]thiophen-5-ylmethanesulfonyl)[2-(2.5-dioxo-imidazolidin-1-yl)ethyl]-amino}-4-methylpentanoic acid (0.062 g) was dissolved in dry DCM (2.5 ml) and cooled to 0° C. under argon. Oxalyl chloride (0.014 ml) was then added, dropwise, followed by DMF (0.011 ml) added dropwise. The resultant mixture was stirred at 0° C. for 30 min. In a separate flask. hydroxylamine hydrocloride (0.037 g) was stirred in THF/water (4 ml THF, 1 ml water) at 0° C. Diethylaminomethyl polystyrene resin (3.2 mmol/g, 0.501 g) was then added and the reaction stirred at 0° C. for 20 min. The solution of acid chloride was then added to the hydroxylamine solution, dropwise at 0° C. The reaction was allowed to warm to rt and stirred overnight. The reaction mixture was filtered, the residue washed with methanol. and the filtrate treated with a further quantity of diethylaminomethyl polystyrene (0.50 g) and stirred for 5 min and filtered. The filtrate was then passed through an SCX column (2 g) and the eluent evaporated to a pale yellow solid which was triturated with ether to give the title compound as a pale yellow powder (0.052 g). MS electrospray (+ve ion) 505 (M+Na). ¹H NMR δ (CD₃OD): 7.90 (2H, m), 7.62 (1H, m), 7.40 (2H, m), 4.49 (2H, Abq), 4.11 (1H, m). 3.90 (2H, s), 3.82 (2H, m), 3.60−3.39 (2H, m), 1.82 (1H, m), 1.63−1.52 (2H, m), 0.97−0.83(7H, m). Chiral HPLC single peak RT=12.5 min.

### EXAMPLE 17: (R)-2-{(Benzo[b]thiophen-5-ylmethanesulfonyl)[2-(3-methyl-2,5-dioxo-imidazolidin-1-yl)ethyl]amino}-N-hydroxy-2-phenylacetamide

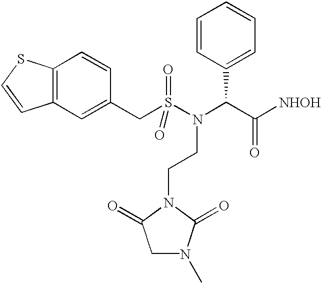

Step 1: (R)-2-(Benzo[b]thiophen-5-ylmethanesulfonylamino)-2-phenylacetic acid tert-butyl ester—The subtitle compound was prepared from (R)-2-(benzo[b]thiophen-5-ylmethanesulfonylamino)-9-phenylacetic acid using the procedure described in Example 1, step 2.

Step 2: (R)-2-[(2-Allyloxyethyl)(benzo[b]thiophen-5-ylmethanesulfonyl)-amino]-2-phenylacetic acid tert-butyl ester—The subtitle compound was prepared from (R)-2-(benzo[b]thiophen-5-ylmethanesulfonylamino)-2-phenylacetic acid tert-butyl ester using the procedure desribed in Example 16, step 2.

Step 3: (R)-2-[(Benzo[b]thiophen-5-ylmethanesulfonyl)(2-hydroxyethyl)amino]-2-phenylacetic acid tert-butyl ester—The subtitle compound was prepared from (R)-2-[(2-allyloxyethyl)(benzo[b]thiophen-5-ylmethanesulfonyl)amino]-2-phenylacetic acid tert-butyl ester using the procedure described in Example 16, step 3.

Step 4: (R)-2-{(Benzo[b]thiophen-5-ylmethanesulfonyl)[2-(3-methyl-2,5-dioxo-imidazolidin-1-yl)ethyl[amino}-N-hydroxy-2-phenylacetamide. (R)-2-[benzo[b]thiophen-5-ylmethanesulfonyl)(2-hydroxyethyl)amino]-2-phenylacetic acid tert-butyl ester was converted into (R,S)-2-{(benzo[b]thiophen-5-ylmethanesulfonyl)[2-(3-methyl-2,5-dioxo-imidazolidin-1-yl)ethyl]amino}-N-hydroxy-2-phenylacetamide using the procedures described in Example 16, steps 4,5, and 6. The racemic compound was separated into component enantiomers by preparative chiral HPLC. Isolation of the faster running isomer afforded the title compound as a white solid. (0.033 g from 0.11 5 g of racemate). MS electrospray (+ve ion) 539 (M+Na). ¹H NMR δ (CD₃OD): 7.93 (2H, m). 7.61 (1H, d, J=5.48), 7.46 (2H, m), 7.39 (5H, m), 5.39 (1H, s), 4.56 (2H, ABq), 3.72 (2H, m), 3.70−3.45 (2H, m), 3.29 (2H, m), 2.86 (3H, s). Chiral purity 99.8% ee.

The following examples were obtained using the procedures described in Example 17. Racemic product was separated by preparative chiral HPLC. In each case isolation of the faster running component afforded the title compound. ¹H NMR and mass spectra were consistent with the structures shown below.

### EXAMPLE 18

### (R)-2-{(Benzo[b]thiophen-5-ylmethanesulfonyl)[2-(2,5-dioxo-imidazolidin-1-yl)ethyl]amino}-N-hydroxy-2-phenyl-acetamide

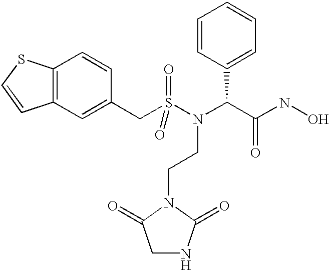

### EXAMPLE 19

### (R)-2-{(Benzo[b]thiophen-5-ylmethanesulfonyl)[2-{(3,4,4-trimethyl)-2,5-dioxo-imidazolidin-1-yl)]ethyl]amino}-N-hydroxy-2-phenyl-acetamide

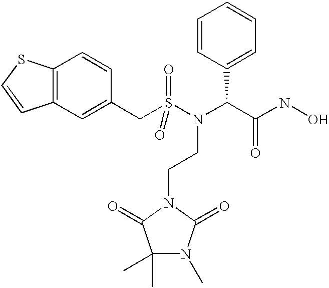

Abbreviations

Bond Elut PSA bonded silica supplied by Varian

BSTFA—Bis trimethylsilyltrifluoroacetamide

DCM—Dichloromethane

DEAD—Diethylazodicarboxylate

Diethylamino Polystyrene resin supplied by FLUKA

DMF—N,N-Dimethylformamide

EDC—1-(3-dimethylaminopropyl)-3-ethylcarbodiimide hydrochloride

EDC—methiodide -1-(3 -dimethylaminopropyl)-3-ethylcarbodiimide methiodide

HOAT—1-Hydroxy-7-azabenzotriazole

rt—Room temperature

SAX—Bonded silica anion exchange support supplied by Varian

SCX—Bonded silica cation exchange support supplied by Varian

TBAF—Tetra-n-butylammonium fluoride

TFA—Trifluoroacetic acid

THF—Tetrahydrofuran

Analytical chiral HPLC analysis was carried out on a Chiralpak AD column using n-hexane/ethanol 50:50 v/v as eluant at a flow rate of 1 ml/min. Preparative chiral HPLC separations were carried out on a Chiralpak AD 250 mm×20 mm i.d. column, using n-hexane/ethanol 50:50 v/v as eluant at a flow rate of 17.0 ml/min.

1. A compound of formula (I): 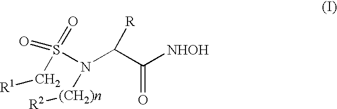
wherein
R is hydrogen, alkyl, alkenyl, alkynyl, aryl, heteroaryl or heterocyclyl; R¹ is bicyclyl or heterobicyclyl; R² is aryl, heteroaryl, heterocyclyl, alkoxy, alkyl, aryloxy, hydroxy or optionally substituted amino and n is from 0 to 3; with the provisos that when n is 0 R² is alkyl or when n is from 1 to 3 R² is not alkyl.

2. A compound according to claim 1 wherein R is isobutyl, phenyl or cyclohexyl, and/or R¹ is 2-naphthyl, 5-benzothiophene, 2-tetrahydronaphthyl or 2-indanyl and/or R² is 2-methoxyethoxy, pyridin-3-yl, furan-2-yl, 5-dimethylaminofuran-2-yl, 2,5-dioxoimidazolidin-1-yl, 3-methyl-2,5-dioxoimidazolidin-1-yl, 3,4,4-trimethyl-2,5-dioxoimidazolidin-1-yl, N-morpholino, N,N-dimethylamino, N-succinimido, N-pyrrolidone or phenoxy and n is 1 or 2.

3. A compound selected from the group consisting of
(R)-2-{(Benzo[b]thiophen-5-ylmethanesulfonyl)[2-(2-methoxyethoxy)-ethyl]amino}-N-hydroxy-2-phenylacetamide;
(R,S)-2-[(Benzo[b]thiophen-5-ylmethanesulfonyl)(pyridin-3 -ylmethyl)-amino]-N-hydroxy-2-phenylacetamide;
(R)-2-[(Benzo[b]thiophen-5-ylmethanesulfonyl)(furan-2-ylmethyl)-amino]-2-cyclohexyl-N-hydroxy-acetamide;
(R)-N-Hydroxy-4-methyl-2-[(naphthalen-2-ylmethanesulfonyl)(pyridin-3-ylmethyl)amino]pentanoic acid amide;
(R)-2-{(Benzo[b]thiophen-5-ylmethanesulfonyl)[2-(2,5-dioxo-imidazolidin-1-yl)ethyl]amino}-4-methyl-N-hydroxy-pentanamide;
(R)-2-{(Benzo[b]thiophen-5-ylmethanesulfonyl)[2-(3-methyl-2,5-dioxo-imidazolidin-1-yl)ethyl]amino}-N-hydroxy-2-phenylacetamide;
(R)-2-{(Benzo[b]thiophen-5-ylmethanesulfonyl)[2-(2,5-dioxo-imidazolidin-1-yl)ethyl]amino}-N-hydroxy-2-phenyl-acetamide;
(R)-2-{(Benzo[b]thiophen-5-ylmethanesulfonyl)[2-[(3,4,4-trimethyl)-2,5-dioxo-imidazolidin-1-yl)]ethyl]amino}-N-hydroxy-2-phenyl-acetamide;
(R)-2-{(Benzo[b]thiophen-5-ylmethanesulfonyl)-[2-(2-methoxyethoxy)ethyl]-amino4-methyl-N-hydroxy-pentanamide;
(R,S)-2-{(Benzo[b]thiophen-5-ylmethanesulfonyl)-[2-(morpholin-4-yl)-ethyl)]-amino}-N-hydroxy-2-phenylacetamide;
(R,S)-2-[(Benzo[b]thiophen-5-ylmethaesulfonyl)-(2-dimethylaminoethyl)-amino]-N-hydroxy-2-phenylacetamide;
(R,S)-2-{(Benzo[b]thiophen-5 -ylmethanesulfonyl)-[2-(2,5-dioxopyrrolidin-1-yl)ethyl]-amino-N-hydroxy-2-phenylacetamide;
(R)-2-{(Benzo[b]thiophen-5-ylmethanesulfonyl)-[2-(2,5-dioxopyrrolidin-1-yl)ethyl]-amino-4-methyl-N-hydroxy-pentanamide;
(R)-2-[(Benzo[b]thiophen-5-ylmethanesulfonyl)-(pyridin-3-ylmethyl)-amino]-4-methyl-N-hydroxy-pentanamide;
(R)-2-{(Benzo[b]thiophen-5-ylmethanesulfonyl)-[2-(2-oxopyrrolidin-1-yl)-ethyl]-amino}-4-methyl-N-hydroxy-pentanamide;
(R)-2-[(Benzo[b]thiophen-5-ylmethanesulfonyl)-(5-dimethylaminomethylfuran-2-ylmethyl)-amino]-4-methyl-N-hydroxy-pentanamide;
(R,S)-2-[(Benzo[b]thiophen-5-ylmethanesulfonyl)-(5 -dimethylaminomethylfuran-2-ylmethyl)-amino]-N-hydroxy-2-phenylacetamide;
(R,S)-2-{(Benzo[b]thiophen-5-ylmethanesulfonyl)-[2-(2-oxo-pyrrolidin-1-yl)-ethyl]-amino}-N-hydroxy-2-phenylacetamide; and
(R,S)-2-[(Benzo[b]thiophen-5 -ylmethanesulfonyl)-(2-phenoxyethyl)-amino]-N-hydroxy-2-phenylacetamide

4. Use of a compound according to any preceding claim for the production of a medicament of the treatment or prophylaxis of disorders in which the overproduction of s-CD23 is implicated.

5. A method for the treatment or prophylaxis of disorders in which the overproduction of s-CD23 is implicated, which method comprises the administration of a compound according to any one of claims 1 to 3 to a human or non-human mammal in need thereof.

6. A pharmaceutical composition for the treatment or prophylaxis of disorders in which the overproduction of s-CD23 is implicated which comprises a compound according to any one of claims 1 to 3 and optionally a pharmaceutically acceptable carrier therefor.

7. Use of a compound according to any one of claims 1 to 3 for the production of a medicament for the treatment or prophylaxis of conditions mediated by TNF.

8. A method for the treatment or prophylaxis of conditions mediated by TNF, which method comprises the administration of a compound according to any one of claims 1 to 3 to a human or non-human mammal in need thereof.

9. A pharmaceutical composition for the treatment or prophylaxis of conditions mediated by TNF, which comprises a compound according to any one of claims 1 to 3 and optionally a pharmaceutical acceptable carrier therefor.

10. A process for preparing a compound according to any one of claims 1 to 3 which process comprises
(a) deprotecting a compound of formula (II): 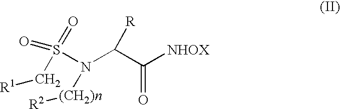
wherein
R, R¹, R² and n are as defined hereinabove, and X is a protecting group such as benzyl, t-butyldimethylsilyl or trimethylsilyl, or
(b) reacting a compound of formula (III): 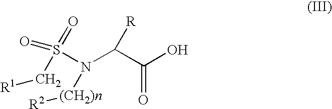
wherein
R, R¹, R² and n are as defined hereinabove, with hydroxylamine or a salt thereof, or
(c) converting a compound of formula (I) to a different compound of formula (I) as defined hereinabove.

11. A compound of formula (II): 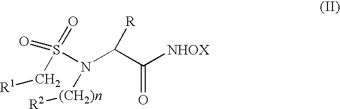
wherein
R, R¹, R² and n are as defined hereinabove.

12. A compound of formula (III): 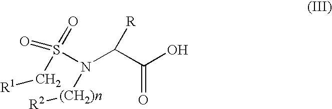
wherein
R, R¹, R² and n are as defined hereinabove.
